(19) United States
(12) Patent Application Publication
(10) Pub. No.: US 2008/0177295 A1
(43) Pub. Date: Jul. 24, 2008

(54) Surgical device having trocar and associated methods

(76) Inventors: Dario Vitali, New Smyrna Beach, FL (US); Rogelio Insignares, Miami, FL (US); Brent Erik Van Camp, Kernersville, NC (US)

Correspondence Address: MYERS & KAPLAN;INTELLECTUAL PROPERTY LAW, L.L.C., CUMBERLAND CENTER II, 3100 CUMBERLAND BLVD , SUITE 1400, ATLANTA, GA, 30339, US

(21) Appl. No.: 11/824,602

(22) Filed: Jun. 28, 2007

Related U.S. Application Data
(60) Provisional application No. 60/817,308, filed on Jun. 29, 2006.

(51) Int. Cl. A61B 17/34

(52) U.S. Cl. 606/185

(57) ABSTRACT

A surgical device having a trocar, a handle with hand-gripping portion adjacent a proximal end, a cannula-interface portion adjacent a distal end and having a recess therein, and an instrument opening extending into a medial portion and through the recess in the cannula-interface portion. The device also includes an instrument seal having an opening therein, positioned in the medial portion of the handle adjacent the instrument opening, a tubular trocar having a proximal end portion adapted to be positioned in the recess of the cannula—interface portion, adjacent the instrument seal and extending outwardly therefrom, and a cannula adapted to receive at least portions of the tubular trocar there through and having a proximal end portion adapted to be received in the recess of the cannula-interface portion of the handle and extending outwardly therefrom so that the handle supports the combination of the cannula and trocar when positioned in the recess.

### CROSS-REFERENCE TO RELATED APPLICATION

To the full extent permitted by law, the present Non-Provisional Patent Application claims priority to and the benefit of United States Provisional patent application entitled “Surgical Device Having Trocar and Associated Methods,” filed on Jun. 29, 2006, having assigned Ser. No. 60/817,308.

### FIELD OF THE INVENTION

The present invention is related to the medical field and, more particularly, to surgical instrumentation having trocars and related methods.

### BACKGROUND OF THE INVENTION

The field of endoscopy requires surgical instruments utilized for minimally invasive surgical procedures. Endoscopic instruments, for example, are used in the medical disciplines of arthroscopy, laparoscopy, rhinoscopy, gynecology, urology, and ENT (ear, nose, and throat). Endoscopic procedures, and the equipment used in connection with endoscopic procedures, affect the quality of patient surgical care.

More specifically, endoscopic procedures and instrumentation affects the way in which patients receive surgery for gallbladder, kidney, hernia, urological, gynecological, cardiovascular heart surgery, and weight loss surgery. Surgeons and healthcare administrators previously have used various instrumentation for the minimally invasive procedures involved in the field of endoscopy. Advancements in instrumentation are needed to improve the quality of such procedures for surgeons and health care administrators.

### SUMMARY OF THE INVENTION

In view of the foregoing, embodiments of the present invention advantageously provide a surgical device having a trocar and methods of using a surgical device to enhance viewing of physical matter. Embodiments of the present invention also advantageously provide a handle, a trocar, and related methods to enhance use of a surgical device and to perform endoscopic and other surgical procedures.

For example, an embodiment of a surgical device having a trocar according to the present invention includes a handle having a hand gripping portion positioned adjacent a proximal end, a cannula-interface portion positioned adjacent a distal end and having a recess therein, and an instrument opening extending into and through a medial portion and also through the recess in the cannula-interface portion. The surgical device also includes an instrument seal having a seal opening and positioned in the medial portion of the handle adjacent the instrument opening to seal instruments when extending through the instrument opening and seal opening. The surgical device also includes a tubular trocar with a proximal end portion adapted to be positioned in the medial portion of the handle adjacent to the instrument opening and that has a distal end portion extending outwardly therefrom through the cannula interface portion. The surgical device further includes a cannula adapted to receive at least portions of the tubular trocar therethrough with a proximal end portion adapted to be received in the recess of the cannula interface portion of the handle and having a distal end portion extending outwardly therefrom so that the handle supports the combination of the trocar and cannula.

Another embodiment of a surgical device according to the present invention includes a handle having a hand-gripping portion positioned adjacent a proximal end, a cannula-interface portion positioned adjacent a distal end, an instrument opening extending into and through a medial portion and the cannula-interface portion thereof. The invention further provides a trocar connected to the handle, the trocar having a proximal end portion with a larger diameter than the distal portion thereof, the proximal end portion of the trocar positioned within the medial portion of the handle adjacent an instrument opening, and the distal end portion of the trocar extending outwardly therefrom.

For example, an embodiment of a handle of a surgical device according to the present invention includes a handle body having a hand-gripping portion positioned adjacent a proximal end of the handle and adapted to be gripped by a hand of a user, a cannula-interface portion positioned adjacent a distal end of the handle adapted to receive and grip a proximal end portion of a cannula therein to thereby allow the cannula to be manipulated by the hand of the user, and an instrument opening extending into and through a medial portion and the cannula-interfaced portion thereof.

Also, for example, an embodiment of a trocar according to the present invention includes a tubular trocar body having a proximal end portion, a distal end portion including an at least optically translucent tip adapted to facilitate the passage of light therethrough, and a longitudinally-extending medial portion connected to and positioned between the proximal end portion and the distal end portion of the tubular trocar body.

The present invention also includes embodiments of using, assemblying, viewing, and positioning a surgical device. For example, an embodiment of a method of using a surgical device includes installing an at least optically translucent tip portion onto a tubular trocar at a distal end of the trocar, mounting a proximal end portion of the tubular trocar within a medial portion of a handle, aligning a tubular cannula to be coaxial with the trocar and a recess of a cannula-interface portion of a handle, sliding a proximal end portion of the cannula over the distal end portion of the trocar so that the tip portion of the trocar at the distal end portion of the trocar extends through a distal end of the cannula, and mounting the cannula within the recess of the cannula-interface portion of the handle.

Another embodiment of a method of using a surgical device includes assembling a trocar and a cannula onto a handle, gripping a gripping-portion of the handle at a position radially offset from an axis of the trocar and cannula, inserting the trocar and cannula into a physical matter, translationally applying a translational force on the handle toward and away from the physical matter in a substantially linear direction along the axis of the trocar and cannula, and rotationally applying a rotational torque on the handle about the axis of the trocar and cannula.

Still another embodiment of a method of using a surgical device to view physical matter outside the surgical device includes assembling a tubular trocar and a tubular cannula into a handle, installing a viewing instrument into and through an instrument opening in the handle and to a hollow cavity in an at least optically translucent distal tip portion of the trocar, and viewing physical matter outside the tip portion of the trocar through the viewing instrument when positioned in the trocar.

### BRIEF DESCRIPTION OF THE DRAWINGS

Some of the features and advantages of the present invention having been stated, others will become apparent as the description proceeds when taken in conjunction with the accompanying drawings, in which:

FIG. 1 is an environmental perspective view of a surgical device after insertion into a type of physical matter according to an embodiment of the present invention;

FIG. 2 is a perspective view of the surgical device of FIG. 1 according to an embodiment of the present invention;

FIG. 3 is a right side elevational view of a surgical device of FIG. 1 according to an embodiment of the present invention;

FIG. 4 is an exploded perspective view of a surgical device of FIG. 1, showing a handle, trocar, and cannula before assembly according to embodiments of the present invention;

FIG. 5 is a sectional view of a surgical device having a trocar and showing the trocar assembled to a handle according to an embodiment of the present invention;

FIG. 6 is an exploded right side lavational view of a surgical device and showing a trocar assembled to a handle, before assembly of a cannula according to an embodiment of the present invention;

FIG. 7 is an exploded environmental view of a surgical device and showing a trocar assembled to a handle, before assembly of a cannula according to an embodiment of the present invention;

FIG. 8 is an exploded environmental view of a surgical device having an instrument inserted therein before insertion into a cannula according to an embodiment of the present invention;

FIG. 9 is a perspective view of a surgical device, including an instrument after assembly into the surgical device according to another embodiment of the present invention;

FIG. 10 is a fragmentary perspective view of a tip of a trocar and a cannula of a surgical device according to an embodiment of the present invention;

FIG. 11 is an exploded perspective view of a tip of a trocar according to an embodiment of the present invention; and

FIG. 12 is an exploded sectional view of a tip of a trocar taken along line 12-12 of FIG. 11 according to an embodiment of the present invention.

### DETAILED DESCRIPTION

The present invention now will be described more fully hereinafter with reference to the accompanying drawings in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

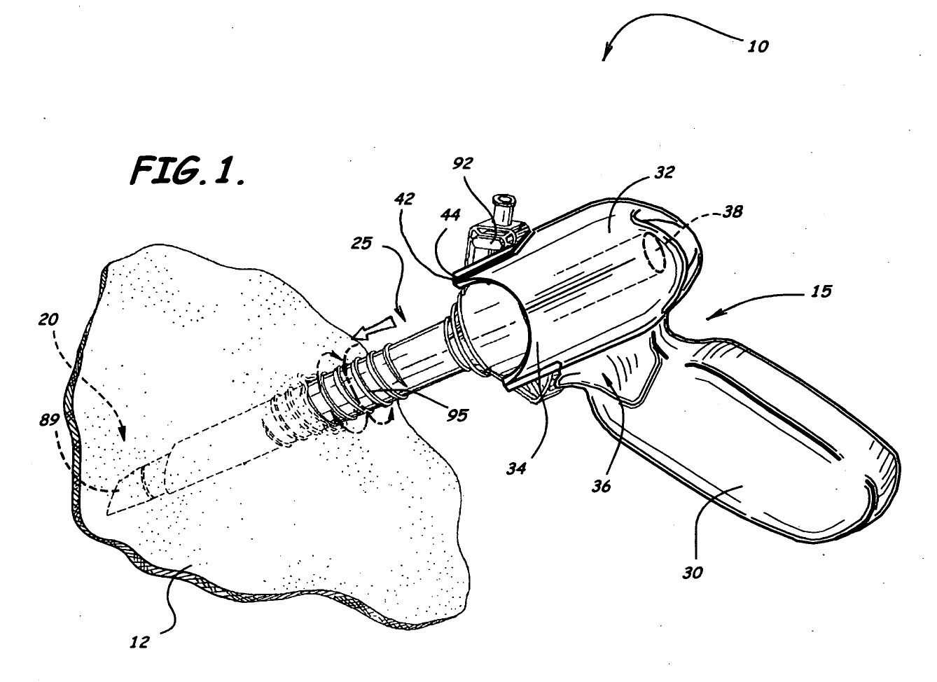

FIGS. 1-3 illustrate an embodiment of a surgical device 10 having a handle 15, a trocar 20, and a cannula 25. Each of the handle 15, trocar 20, and cannula 25 includes a proximal end and a distal end. As shown in FIGS. 1-3, the handle 15 advantageously can support both the trocar 20 and the cannula 25 when the cannula is positioned on the trocar 20. The handle 15 has a hand-gripping portion 30, a medial portion 32, and a cannula-interface portion 34. The handgripping portion 30 is positioned adjacent the proximal end of the handle 15. The hand-gripping portion 30 can include a throat 36 that connects the hand-gripping portion 30 of the handle 15 with the medial portion 32 of the handle 15. When a user, such as a surgeon or other medical practitioner, uses the surgical device 10, the hand of the user wraps around the hand-gripping portion 30 of the handle 15, and optionally one of the fingers of the hand of the user may wrap around the throat 36 of the hand-gripping portion 30 of the handle 15 for more secure handling during operation of the surgical device 10. The hand-gripping portion 30 is shaped in a bulbous and curvaceous manner so that the hand of the user fits comfortably around the hand-gripping portion 30. The throat 36 of the hand-gripping portion 30 can be thinner than the lower part or more proximal portion of the hand-gripping portion 30 to enable the user to securely insert a finger snugly on the thinner throat 36 portion of the hand-gripping portion 30 of the handle 15. The hand-gripping portion 30, for example, can be made of a plastic material or other materials such as rubber, metal, or another suitable material as understood by those skilled in the art, and can include ridges thereon, for example, for easier gripping by the hand of the user.

The throat 36 of the hand-gripping portion 30 of the handle 15 connects the upper part of the hand-gripping portion 30 with the lower part of the medial portion 32 of the handle 15. The hand-gripping portion 30 extends outwardly from or below the medial portion 32 at a preselected angle X from a longitudinally extending axis of the cannula-interface portion 34, e.g., having a handle axis between 90° to 180° to a cannula interface axis, and more preferably between 110° to 150°, of the handle 15. The medial portion 32 has a substantially cylindrical surface portion supported upon the throat 36 of the hand-gripping portion 30 of the handle 15. The medial portion 32 of the handle 15 has an instrument opening 38 at the proximal end to receive surgical instruments, optical viewing instruments such as an endoscope, or other instruments during operation of the surgical device 10. The instrument opening 38 extends into and through the medial portion 32 of the handle 15 and also through the cannula-interface portion 34 of the handle 15.

The cannula-interface portion 34 is positioned adjacent a distal end of the handle 15. The substantially cylindrical surface portion of the medial portion 32 adjoins a substantial cylindrical surface portion of the cannula-interface portion 34. The cannula-interface portion 34 of the handle 15 includes a recess 40 coaxial or coextensive with and distal to the instrument opening 38. The recess 40 has a larger diameter than the instrument opening 38. The recess 40 also has a larger diameter than the proximal portions of both the cannula 25 and the trocar 20. The recess 40 receives the cannula 25 and trocar 20 when they are inserted into the handle 15 for operation of the surgical device 10. The trocar 20 preferably is connected to the handle 15 and the cannula 25 slides onto the trocar 20. The cannula 25, for example, can be a cannula as further described in detail in U.S. Pat. No. 6,569,119 also owned by the common assignee of the present application or a cannula as described in detail in co-pending U.S. patent application Ser. No. 10/879,644 filed on Jun. 29, 2004, which is a continuation of U.S. patent application Ser. No. 10/763,762 filed on Jan. 23, 2004, which is now abandoned, and each of which are incorporated herein by reference in their entireties.

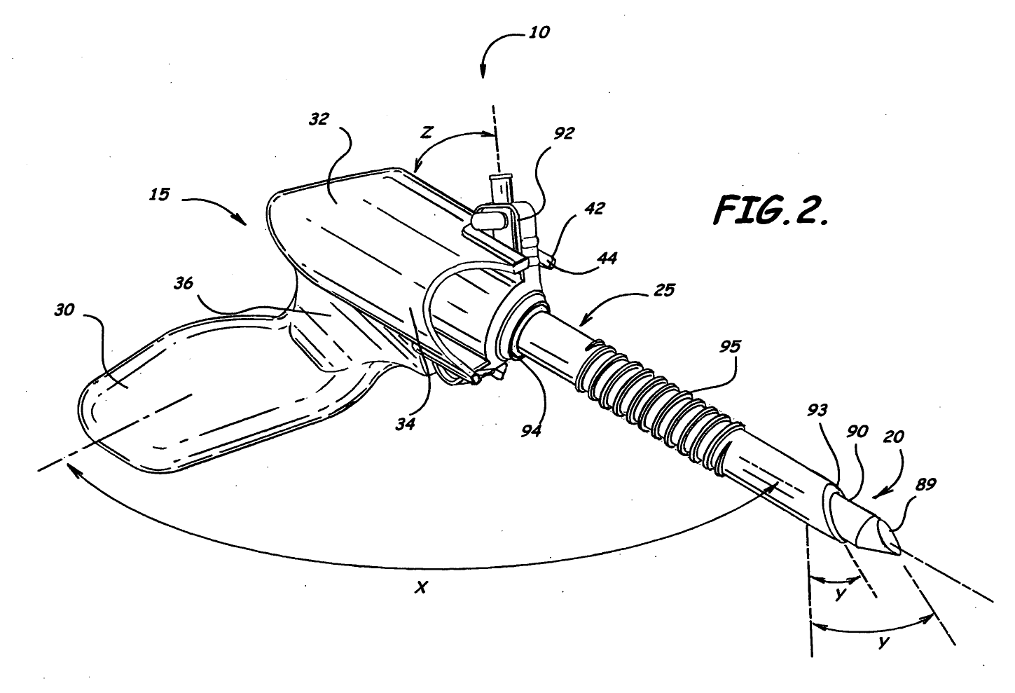

The cannula-interface portion 34 of the handle 15 also, for example, can have a cannula grip 42, such as provided by a pair of circumferentially spaced-apart slots 44 at the distal end of the cannula-interface portion 34. As shown in FIG. 1 and FIG. 2, the slots 44 extend inwardly in the axial direction of the axis of the cannula-interface portion 34. In this embodiment, the pair of slots 44 is advantageously positioned spaced-apart circumferentially 180 degrees from each other, but, as understood by those skilled in the art, other angular positions and types of slots or grips can be used as well according to the present invention. Alternatively, the cannula-interface portion 34 may have more than two slots 44, in which case the slots 44 can be spaced-apart circumferentially at equal angular intervals along the circumference of the cannula-interface portion 34.

The slots 44 of the cannula grip 42 receive and grip outer surface portions of proximal end portions of a cannula 25 therein. The proximal end portion of the cannula 25 is capable of moving translationally or longitudinally toward and away from the cannula grip 42 of the handle 15. The cannula grip 42, however, in this embodiment, prevents or restricts rotational movement of the cannula 25 relative to the cannula grip 42 of the handle 15 when the cannula 25 is being gripped by the cannula grip 42. In this manner, the cannula 25 can be manipulated by a hand of a user when the hand of the user operates the handle 15 of the surgical device 10, and particularly for insertion of the cannula 25 or for stabilizing the cannula 25 for use of an instrument through the instrument seal.

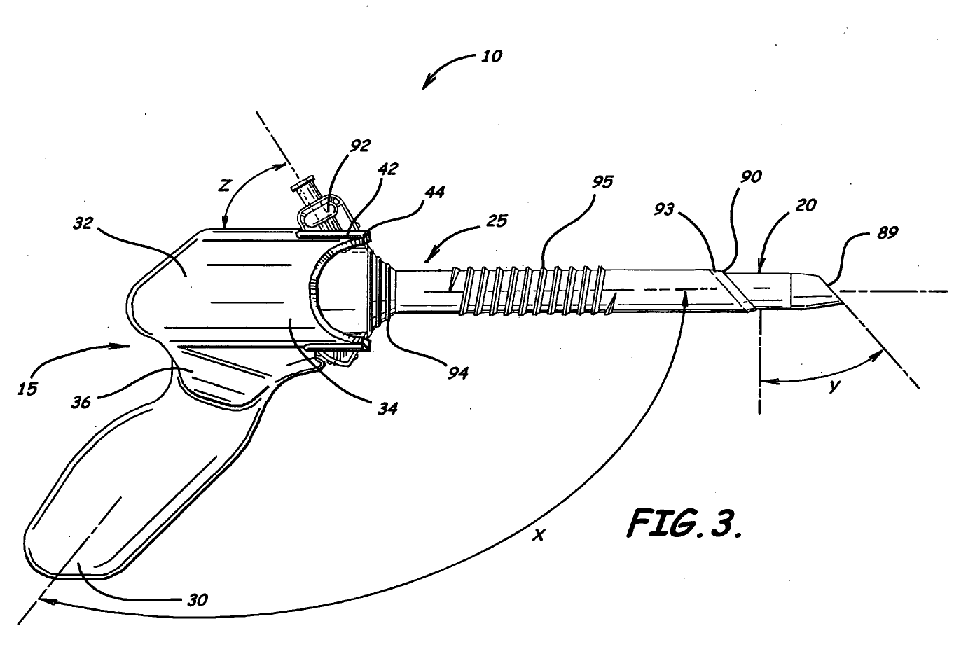

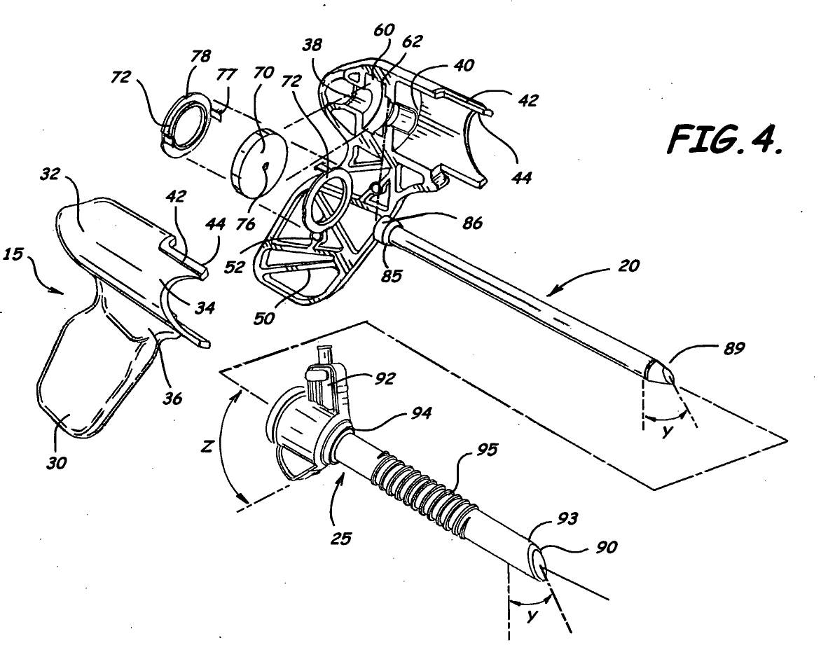

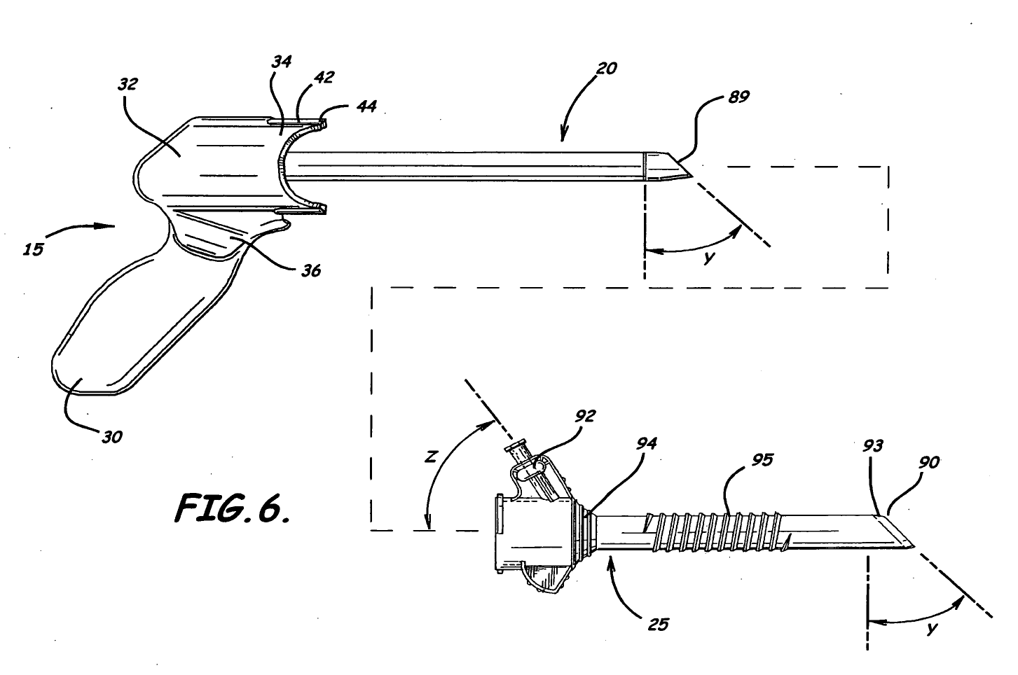

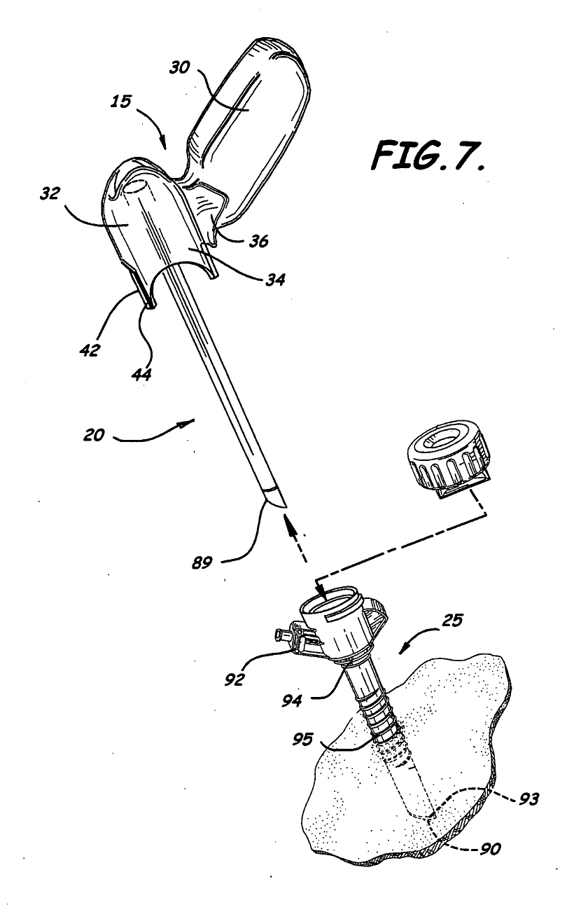

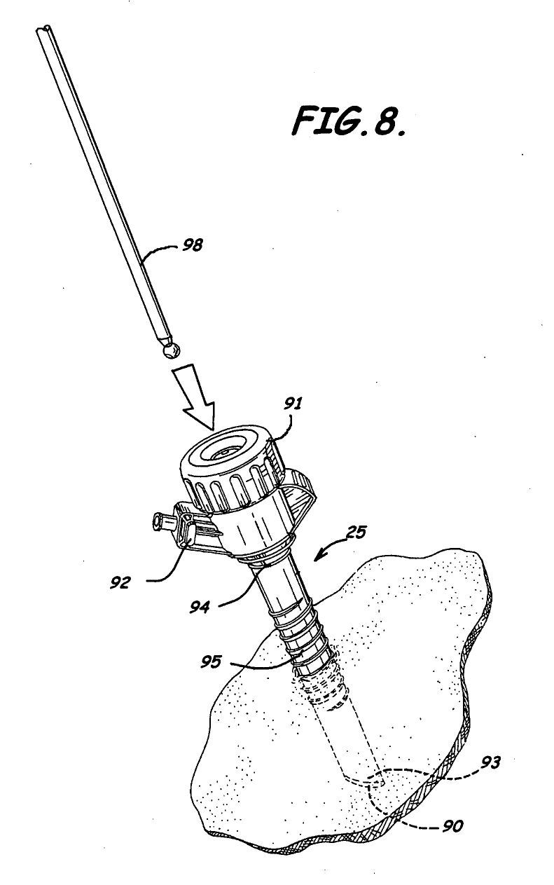

FIGS. 4-9, for example, illustrate exploded views of a surgical device 10, a handle, a trocar 20, and a cannula 25 according to embodiments of the present invention. The handle 15 has a pair of handle-portions, e.g., half-portions, capable of being connected securely together or separating apart. Each half-portion of the handle 15, for example, includes half-portions of the hand-gripping portion 30, the medial portion 32, and the cannula-interface portion 34, as illustrated in FIG. 3. The inner portions of the hand-gripping portion 30 and throat 36 of the handle 15 include a structure of webbed members 50 to support the outer portions of the handgripping portion 30 and throat 36 when the hand of the user grips the handle 15, as well as for securing the instrument seal therein and a proximal end portion of the trocar 20 therein. Several openings 52 or female receptors for receiving fasteners are located within several of the webbed members 50. The fasteners, such as opposing male members, screws, bolts, or other fasteners, fit inside the openings 52 to hold the pair of half-portions of the handle 15 together after assembly and during operation of the surgical device 10. Such openings 52 and fasteners may also be used on other portions of the handle 15 as well, including the inner portions of the medial portion 32 and the cannula-interface portion 34.

The inner portions of the medial portion 32 of the handle 15 include the instrument opening 38 extending therethrough and a half-cylindrical groove 60 substantially parallel and substantially coaxial to the instrument opening 38. An instrument seal groove 60 forms a cylindrical cavity to receive and accommodate an instrument seal 70, as well as a pair of annular seal retaining or locking rings 72 on either opposing face of the instrument seal 70, when the half-portions of the handle 15 are joined together to form a singular handle.

The instrument seal 70 has a seal opening 76 and is positioned in the inner portion of the medial portion 32 of the handle 15 body adjacent and coaxial to the instrument opening 38 to effectively seal and/or position align any instruments that may extend through the instrument opening 38 and seal opening 76. The flexible or expandable portions of the instrument seal 70 immediately surrounding the seal opening 76 are made from a flexible material such as silicone, flexible plastic or rubber, or other stretchable material. When a surgical instrument with a diameter larger than the seal opening 76 extends through the seal opening 76, for example, the flexible material of the instrument seal 70 surrounding the seal opening 76 can expand to the diameter of the surgical instrument extending therethrough. The instrument seal 70 effectively seals foreign substances from passing through the instrument seal 70 when surgical instruments pass through the instrument opening 38 and the instrument seal 70 into the trocar 20.

The pair of annular retaining rings 72 is positioned adjacent and coaxial with the instrument seal 70. The retaining rings 72 face each other on each opposing face of the instrument seal 70 so that the instrument seal 70 is sandwiched in between each of the retaining rings 72. The pair of retaining rings 72 can have latches 77 and latch receivers 78 to connect with each other to hold the instrument seal 70 in between the pair of retaining rings 72. The latches 77 on one of the retaining rings 72, for example, can latch onto the latch receiver 78 on the other retaining ring 72 when the instrument seal 70 is positioned in between the two retaining rings 72. The retaining ring 72 thereby retains or secures the instrument seal 70 within the inner portion of the medial portion 32 of the handle 15 while a surgical instrument extends through the instrument seal 70.

The handle 15 can also have an air pocket groove 62 adjacent the instrument seal groove 60 positioned in each half-portion of the medial portion 32 of the handle 15, if desired. The air pocket groove 62, for example, can be located coaxial and parallel with the instrument seal groove 60. The air pocket groove 62 compliments the instrument seal groove 60 by providing special room for flexure and movement of the instrument seal when positioned adjacent thereto.

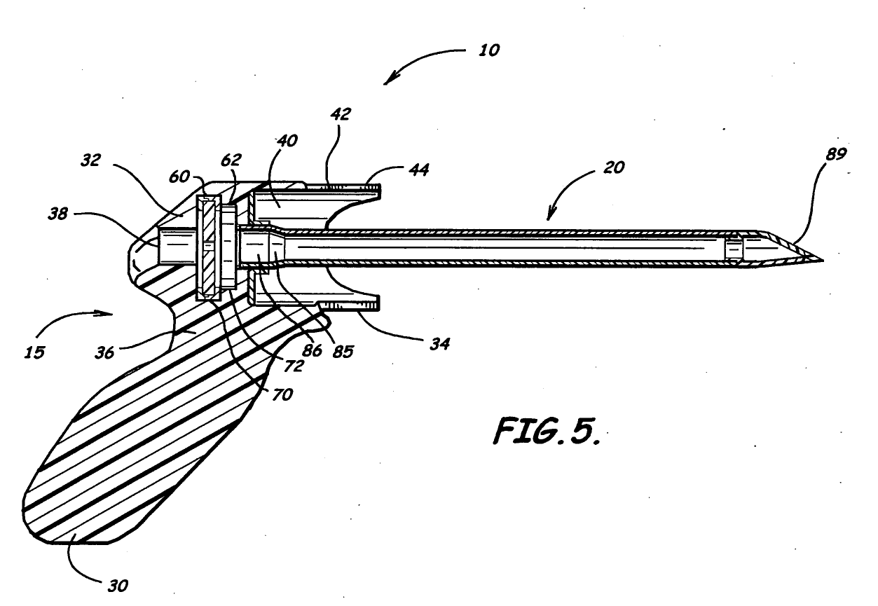

A flange or lip 86 at the proximal end portion of the trocar 20 is contained within the inner portion of the medial portion 32 of the handle 15 adjacent the instrument seal groove 60 and the air pocket groove 62, as shown in FIGS. 4-5. When the pair of half-portions of the handle 15 join together, the half-portions of the handle 15 as joined- collectively encase the instrument seal 70, seal retaining ring 72, and proximal end portion of the trocar 20 within the handle 15.

The trocar 20 is a tubular member formed of a stainless steel, polycarbonate, or other suitable material. The trocar 20 has a lip 86 at the proximal end portion secured within the inner portion of the medial portion 32 of the handle 15 and a distal end portion extending outwardly therefrom through the cannula-interface portion 34. The proximal end portion of the trocar 20 includes a lip 86 extending outwardly from the proximal end portion. The lip 86 is received within the inner portion of the medial portion 32 of the handle 15. A longitudinal portion of the trocar 20 joins the proximal end portion and the distal end portion of the trocar 20. If desired, as understood by those skilled in the art, the proximal end portion of the trocar 20 has a larger diameter or circumference than the longitudinal portion and the distal end portion of the trocar 20.

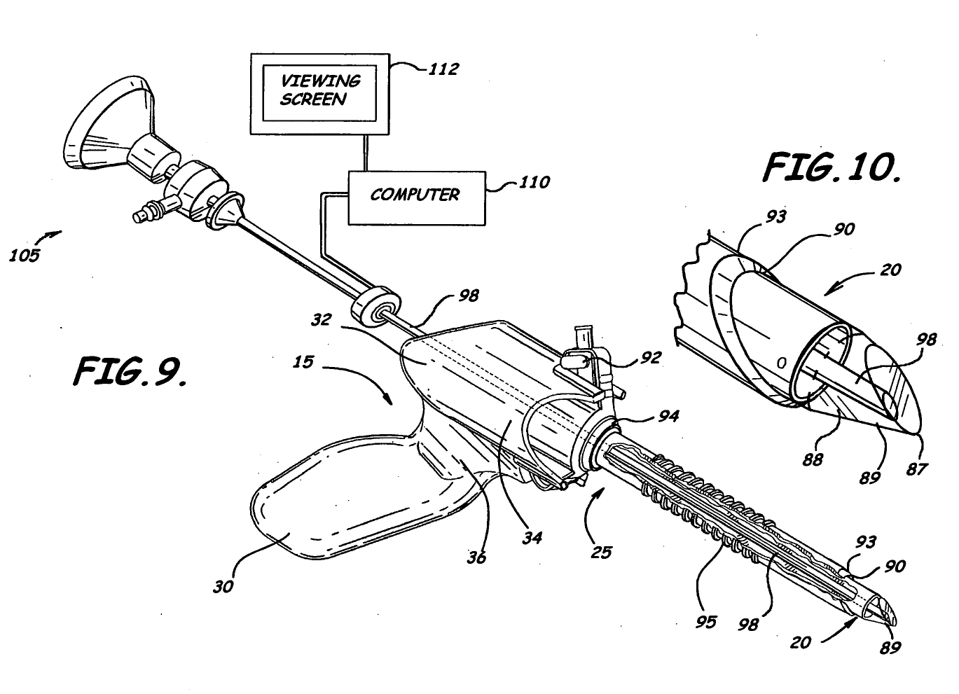

As shown in FIGS. 10-12, the distal end portion of the trocar 20 has an at least optically translucent tip portion 89, or alternatively an optically clear transparent tip portion 89 or an entirely clear opening at the tip portion 89. The tip, for example, can be formed of a polycarbonate material or other material as understood by those skilled in the art. The optically translucent tip portion 89 is capable of facilitating the passage of light from outside the tip portion 89 of the trocar 20 to inside the tip portion 89 of the trocar 20. In the preferred embodiment, the tip portion 89 is made of a plastic material without a discernable blade at the point 87 of the tip portion 89. For example, the point 87 of the tip portion 89 is relatively sharp but will not cut the operator's finger upon simple touching thereof. Alternatively, the point 87 of the tip portion 89 may be made of a sharp metal blade, a blunt rubber edge, or other material depending on the particular purpose or application of the trocar 20.

The tip portion 89 has a hollow cavity 88, and is formed in a conical shape except that a portion of the conical surface is deformed by an angled surface shaped at a preselected angle relative to a plane perpendicular to an axis of the trocar to facilitate insertion. The tip portion 89 is cut at a preselected angle Y relative to a plane perpendicular to an axis of the trocar 20. The angled tip portion 89 is employed to facilitate insertion of the distal end portion of the trocar 20 into a physical matter 12.

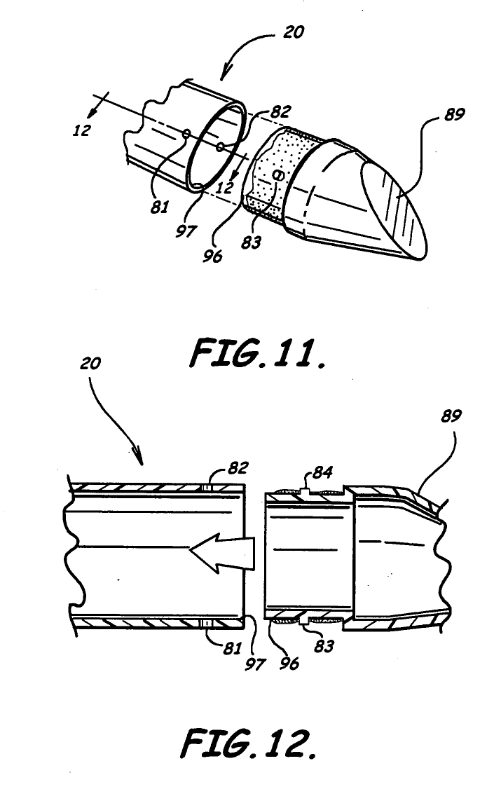

The tip portion 89 has a male portion 96 that is received in the female portion 97 of the longitudinal portion of the trocar 20, as shown in FIGS. 11-12. A pair of nubs 83, 84 protrude from the outer surface of the male portion 96 of the tip portion 89 of the trocar 20. The nubs 83, 84 are circumferentially spaced-apart 180 degrees from each other along the outer surface of the male portion 96. Also, a pair of nub receptacles 81, 82 are defined at the distal end of the female portion 97 longitudinal portion of the trocar 20 to receive the nubs 83, 84 therein when the tip portion 89 is installed on the female portion 97 of the longitudinal portion of the trocar 20. The nubs 83, 84 protrude from the outer surface of the male portion 96 with a protrusion length substantially equal to the wall thickness of the longitudinal portion of the trocar 20, so that the nub receptacles 81, 82 on the female portion 97 have a substantially similar depth as the protrusion length of the nubs 83, 84.

To install the male portion 96 of the tip portion 89 to the female portion 97 of the longitudinal portion of the trocar 20, the user squeezes the male portion 96 so that the nubs 83, 84 can fit inside the tubular female portion 97 of the longitudinal portion of the trocar 20. When the user aligns the nubs 83, 84 with the nub receptacles 81, 82, the nubs 83, 84 fit, are positioned, or snap into the nub receptacles 81, 82, thereby enhancing a firmly secured tip portion 89 of the trocar 20. Additionally, the male portion 96 and the female portion 97 also feature an orientation key to align the tip portion 89 of the trocar 20 with the longitudinal portion of the trocar 20. Also, additionally, an adhesive, such as glue, epoxy, or other adhesive substance, is applied between the male portion 96 of the tip portion 89 of the trocar 20 and the female portion 97 of the longitudinal portion of the trocar 20 to further improve stability and strength of the tip portion 89 of the trocar 20.

A tubular portion 90 of the cannula 25 receives at least portions of the tubular trocar 20 therethrough, as illustrated in, for example, FIGS. 4-7. The trocar 20 can slide into the cannula 25 by inserting the distal end portion of the trocar 20 into the proximal end portion of the cannula 25. The trocar 20 can slide out of the cannula 25 by removing the distal end portion of the trocar 20 from the proximal end portion of the cannula 25. The inner diameter of the tubular portion 90 of the cannula 25 has a substantially similar diameter as the outer diameter of the tubular trocar 20. The cannula 25 has a longitudinal extent shorter than the longitudinal extent of the trocar 20. The trocar 20 slides through the tubular portion 90 of the cannula 25 until the proximal end portion of the trocar 20 is flush with the inside of the proximal end portion of the cannula 25, so that at least a portion of the tip portion 89 at the distal end portion of the trocar 20 extends outwardly farther than a distal end portion of the cannula 25 when having the trocar 20 fully extended through the cannula 25.

The cannula 25 has a proximal end portion received in the recess 40 of the cannulainterface portion 34 of the handle 15. The distal end portion of the cannula 25 extends outwardly from the cannula-interface portion 34 of the handle 15, thereby causing the handle 15 to support the weight of the combination of the trocar 20 and cannula 25. The pair of slots 44 of the cannula grip 42 on the cannula-interface portion 34 of the handle 15 receive a pair of wings 92 on the proximal end portion of the cannula 25. The wings 92 of the cannula 25 have a substantially similar depth and width as the slots 44 on the cannula grip 42. The pair of wings 92 is circumferentially spaced-apart along the circumference of the proximal end portion of the cannula 25. As shown in FIGS. 2-3, the wings 92 extend outwardly from the proximal end portion of the cannula 25 at a preselected angle Z relative to the longitudinal axis of the cannula 25. The wings 92 are gripped by the slots 44 of the cannula grip 42 at the preselected angle Z. The wings 92 slideably engage the corresponding slots 44 of the cannula grip 42 on the handle 15, so that the wings 92 are securely gripped by the cannula grip 42. The pair of wings 92 is spaced-apart circumferentially 180 degrees from each other. Alternatively, the proximal end portion of the cannula 25 may have more than two wings 92, in which case the wings 92 would be spaced-apart circumferentially at equal angular intervals along the circumference of the proximal end portion of the cannula 25.

A longitudinal portion of the cannula 25 joins the proximal end portion and the distal end portion of the cannula 25. The proximal end portion of the cannula 25 has a larger diameter or circumference than the longitudinal portion and the distal end portion of the cannula 25. A cannula chamfer 94 adjacent the proximal end portion of the cannula 25 joins the proximal end portion of the cannula 25 with the longitudinal portion of the cannula 25. The cannula chamfer 94 may have a ribbed portion. The distal edge 93 of the cannula 25 is cut at the same preselected angle Y as the tip portion 89 of the trocar 20 at the distal end portion of the trocar 20. The angle Y of the distal edge 93 of the cannula 25 is relative to a plane perpendicular to an axis of the trocar 20. The angled distal edge 93 is employed to facilitate insertion of the distal end portion of the cannula 25 into a physical matter 12. A helical rib 95 protrudes from an outer surface portion of the longitudinal portion of the cannula 25 to prevent inadvertent retraction of the cannula 25 when inserted into a physical matter 12.

As shown in FIG. 1, in operation, the cannula can move in either rotational direction and can move in either translational direction within the physical matter, based upon the relative positioning of the cannula and trocar desired by the user. The cannula 25 may be used in operation in conjunction with the handle 15, or alternatively with the handle 15 detached from the cannula 25. When the cannula 25 is removed or separated from the cannula-interface portion 34 of the handle 15, a cannula cap 91 is placed on the cannula 25 to prevent foreign substances from penetrating the inside portions of the cannula 25.

Many instruments 98 may be installed through the instrument opening 38 of the handle 15 and into the tubular trocar 20 to the tip portion 89 of the distal end portion of the trocar 20. For example, a surgical instrument 98 may be installed through the instrument opening 38 and into the tip portion 89 of the trocar 20 for viewing a surgical operation beyond the tip portion 89 of the trocar 20 within the physical matter 12. The surgical instrument 98 may be connected outside the instrument opening 38 of the handle 15 to a computer system 110 or a viewing screen 112 to better assist the operator in viewing surgical operations in the physical matter 12.

Additionally, for example, in conjunction with the surgical instrument 98 or independent of the surgical instrument 98, an optical instrument, viewing instrument, or endoscope 105 may be installed into the instrument opening 38 and into the trocar 20 for viewing the surgical instrument's 98 relative position inside the physical matter 12, or for simply viewing the physical matter 12 independent of the surgical instrument 98. A surgical instrument 98 also can be connected outside the instrument opening 38 of the handle 15 to a computer system 110 or a viewing screen 112 such as a monitor or a television screen. The insides of the physical matter 12 may be communicated through the surgical instrument 98 into the computer system 110 or viewing screen 112, so that the user may view the relative status of the inside portion of the physical matter 12 outside the tip portion 89 of the trocar 20.

The tip portion 89 of the trocar 20 can be a substantially closed or fully-closed/sealed as illustrated in the drawings herein. In this case, as illustrated in the drawings, a surgical instrument 98 such as a scope or endoscope may be installed to extend to the tip portion 89 of the trocar 20 so that the surgical instrument 98 does not have access to the physical matter 12. An optical viewing instrument 98 that views the operation through the tip portion 89 of the trocar 20 may be utilized in conjunction with another surgical instrument 98 that extends through an opening in the cannula after removal of the trocar from the cannula for operation on physical matter 12.

In operation, as shown in FIG. 1, the handle 15 can supports both the trocar 20 and the cannula 25. The user operates the surgical device 10 by holding the gripping-portion of the handle 15 and moving the surgical device 10 translationally and rotationally, in order to enter a physical matter 12 for surgical operation within the physical matter 12. An instrument 98, as shown in FIG. 9, may be installed within the surgical device 10 for viewing the physical matter 12 through the optically translucent or transparent tip portion 89 of the trocar 20.

As illustrated in FIGS. 1-12 and as described above herein, the present invention also includes embodiments of using, assemblying, viewing, and positioning a surgical device. For example, an embodiment of a method of using a surgical device 10 includes installing an at least optically translucent tip portion onto a tubular trocar 20 at a distal end of the trocar, mounting a proximal end portion of the tubular trocar 20 within a medial portion of a handle, aligning a tubular cannula 25 to be coaxial with the trocar 20 and a recess of a cannula-interface portion of a handle, sliding a proximal end portion of the cannula 25 over the distal end portion of the trocar 20 so that the tip portion of the trocar 20 at the distal end portion of the trocar extends through a distal end of the cannula 25, and mounting the cannula 25 within the recess of the cannula-interface portion of the handle. The method can also include installing a surgical instrument into and through the handle and also through at least portions of the tubular trocar 20 to optically view physical matter outside the at least optically translucent tip portion of the trocar 20, providing a substantially conical surface on the tip portion of the trocar 20, and deforming the conical surface by a flat angled surface shaped at a preselected angle relative to a plane perpendicular to an axis of the trocar 20. The at least optically translucent tip portion, for example, can be selected from the group consisting of: optically translucent tip, optically transparent tip, and optically clear tip. The method can further include installing an instrument seal having a seal opening in the medial portion of the handle to seal instruments when extending therethrough, installing a seal retaining ring adjacent the instrument seal to assist in retaining the instrument seal within the handle, and receiving a wing of the cannula 25 within a slot of the cannula-interface portion of the handle to assist in restricting rotational movement of the cannula 25 relative to the handle.

Another embodiment of a method of using a surgical device 10 includes assembling a trocar 20 and a cannula 25 onto a handle, gripping a gripping-portion of the handle at a position radially offset from an axis of the trocar 20 and cannula 25, inserting the trocar 20 and cannula 25 into physical matter, translationally applying a translational force on the handle toward and away from the physical matter in a substantially linear direction along the axis of the trocar 20 and cannula 25, and rotationally applying a rotational torque on the handle about the axis of the trocar 20 and cannula 25. The method can also include the assembling including arranging the trocar 20 within a tubular portion of the cannula 25 in a coaxial disposition, the translationally applying further including moving the trocar 20 and cannula 25 into and out of the physical matter, and the rotationally applying further including rotating the trocar 20 and cannula 25 within the physical matter clockwise and counterclockwise in a circular direction.

Still another embodiment of a method of using a surgical device to view physical matter outside the surgical device 10 includes assembling a tubular trocar 20 and a tubular cannula 25 into a handle, installing a viewing instrument into and through an instrument opening in the handle and to a hollow cavity in an at least optically translucent distal tip portion of the trocar 20, and viewing physical matter outside the tip portion of the trocar 20 through the viewing instrument when positioned in the trocar 20. The method can also include interfacing the viewing instrument with a computer system and communicating the viewing of the physical matter to a computer system interfaced with the viewing instrument. The method additionally can include interfacing the viewing instrument with a viewing screen and communicating the viewing of the physical matter to a viewing screen interfaced with the viewing instrument. The method can further include translationally moving the surgical device 10 and the viewing instrument within the physical matter in a linear direction along the axis of the trocar 20 and cannula 25 to obtain different views of the physical matter from different positions within the physical matter. The method still further can include rotationally moving the surgical device 10 and the viewing instrument within the physical matter in a rotational direction about the axis of the trocar 20 and cannula 25 to obtain different views of the physical matter from different positions within the physical matter.

In the drawings and specification, there have been disclosed typical preferred embodiments of the invention and, although specific terms are employed, they are used in a generic and descriptive sense only and not for the purpose of limitation, the scope of the invention being set forth in the following claims.

1. A surgical device comprising:
a handle having a hand-gripping portion positioned adjacent a proximal end, a cannula-interface portion positioned adjacent a distal end and having a recess therein, and an instrument opening extending into and through a medial portion and also through the recess in the cannula-interface portion;
an instrument seal having a seal opening and positioned in the medial portion of the handle adjacent the instrument opening, to seal instruments when extending through the instrument opening and seal opening;
a tubular trocar having a proximal end portion adapted to be positioned in the medial portion of the handle adjacent the instrument opening, and having a distal end portion extending outwardly therefrom through the cannula-interface portion; and
a cannula adapted to receive at least portions of the tubular trocar therethrough, having a proximal end portion adapted to be received in the recess of the cannula-interface portion of the handle, and having a distal end portion extending outwardly therefrom so that the handle supports the combination of the trocar and cannula.

2. A device as defined in claim 1, further comprising a seal retaining ring positioned adjacent the instrument seal to assist in retaining the instrument seal within the handle, wherein the proximal end portion of the trocar has a larger circumference than the distal portion thereof, and wherein the proximal end portion of the cannula has a larger circumference than the distal portion thereof.

3. A device as defined in claim 1, wherein the trocar tip is formed of a rigid polymeric material and further has an at least optically translucent tip at the distal end portion thereof adapted to facilitate passage of light therethrough, and wherein the cannula has a longitudinal extent shorter than a longitudinal extent of the trocar so that at least a portion of the tip of the trocar extends outwardly farther than a distal end of the cannula when having the trocar extended therethrough.

4. A device as defined in claim 3, wherein the at least optically translucent tip of the trocar and the distal end of the cannula are formed at a preselected angle relative to a plane perpendicular to an axis of the trocar to facilitate insertion of the trocar.

5. A device as defined in claim 1, wherein the cannula-interface portion of the handle includes a cannula grip to grip a proximal end portion of the cannula when positioned in the cannula-interface to thereby allow the cannula to be manipulated by a hand of a user when gripping the hand-gripping portion of the handle.

6. A device as defined in claim 5, wherein the cannula grip includes a pair of circumferentially spaced-apart slots adapted to receive outer surface portions of the proximal end portions of the cannula therein to thereby allow translational movement of the proximal end portions of the cannula toward and away from the cannula grip and to restrict rotational movement of the proximal end portions of the cannula relative to the cannula grip when the cannula is positioned in the cannula grip.

7. A device as defined in claim 6, wherein the proximal end portions of the cannula includes a pair of circumferentially spaced-apart wings extending outwardly from the proximal end portion thereof and adapted to slideably engage the pair of slots of the cannula grip.

8. A device as defined in claim 7, wherein the wings of the cannula extend outwardly from the proximal end portion of the cannula at a preselected angle relative to the longitudinal axis of the cannula, and wherein the wings of the cannula are adapted to be gripped by the slots of the cannula grip of the handle at the preselected angle.

9. A device as defined in claim 1, further comprising a helical rib protruding from an outer portion of the cannula to prevent inadvertent retraction of the cannula when inserted into physical matter.

10. A device as defined in claim 1, wherein the hand-gripping portion of the handle extends at a preselected angle to the trocar extending outwardly therefrom.

11. A surgical device comprising:
a handle having a hand-gripping portion positioned adjacent a proximal end, a cannula-interface portion positioned adjacent a distal end, an instrument opening extending into and through a medial portion and the cannula-interface portion thereof;
a trocar connected to the handle, the trocar having a proximal end portion with a larger diameter than the distal portion thereof, the proximal end portion of the trocar positioned within the medial portion of the handle adjacent an instrument opening, and the distal end portion of the trocar extending outwardly therefrom.

12. A device as defined in claim 11, further comprising an instrument seal having a seal opening and positioned in the medial portion of the handle adjacent the instrument opening, to seal instruments when extending through the instrument opening and seal opening.

13. A device as defined in claim 12, further comprising a seal retaining ring positioned adjacent the instrument seal to assist in retaining the instrument seal within the handle.

14. A device as defined in claim 11, wherein the trocar is formed of a polycarbonate material and further has an at least optically translucent tip at the distal end portion thereof adapted to facilitate passage of light therethrough.

15. A device as defined in claim 14, wherein the at least optically translucent tip of the trocar is formed at a preselected angle relative to a plane perpendicular to an axis of the trocar to facilitate insertion of the trocar.

16. A device as defined in claim 11, wherein the cannula interface portion of the handle includes a cannula grip adapted to grip a proximal end portion of a cannula when positioned in the cannula-interface portion to thereby allow the cannula to be manipulated by a hand of a user.

17. A device as defined in claim 16, wherein the cannula grip includes a pair of circumferentially spaced-apart slots adapted to receive outer surface portions of proximal end portions of the cannula therein to thereby allow translational movement of proximal end portions of the cannula toward and away from the cannula grip and to restrict rotational movement of proximal end portions of the cannula relative to the cannula grip when the cannula is positioned in the cannula grip.

18. A device as defined in claim 11, wherein the hand-gripping portion of the handle extends at a preselected angle to the trocar extending outwardly therefrom.

19. A device as defined in claim 12, wherein the handle includes a pair of half-portions capable of joining together, each half-portion of the handle including half-portions of the hand-gripping portion, the medial portion, and the cannula-interface portion, wherein the pair of half-portions of the handle join together to encase therein the instrument seal and a proximal end portion of the trocar.

20. A device as defined in claim 11, wherein the hand- gripping portion of the handle includes a throat connecting the hand-gripping portion with the medial portion of the handle and adapted to receive a finger of the hand of the user therearound.
